(19) United States
(12) Patent Application Publication
(10) Pub. No.: US 2025/0345379 A1
(43) Pub. Date: Nov. 13, 2025

(54) METHODS AND COMPOSITIONS FOR REDUCING BODY WEIGHT IN MAMMALS

(71) Applicants: David Lafond, Galesburg, MI (US); Christine O'Neil, Jupiter, FL (US)

(72) Inventors: David Lafond, Galesburg, MI (US); Christine O'Neil, Jupiter, FL (US)

(21) Appl. No.: 19/201,520

(22) Filed: May 7, 2025

Related U.S. Application Data
(60) Provisional application No. 63/643,492, filed on May 7, 2024.

(51) Int. Cl. A61K 36/03; A61K 31/353; A61K 31/717; A61K 31/723; A61K 31/734; A61K 31/736; A61K 36/24; A61K 38/56

(52) U.S. Cl. CPC A61K 36/03; A61K 31/353; A61K 31/717; A61K 31/723; A61K 31/734; A61K 31/736; A61K 36/24; A61K 38/56

(57) ABSTRACT

Methods and compositions promote weight loss in mammals, such as in humans and/or animals, such as, for example, pets. Specifically, the methods and compositions herein promote weight loss due to the production of appetite-suppressing gut hormones, increased metabolism, reduced fat and carbohydrate absorption, and reduced fat deposition through the synergistic combination of fibers and botanical extracts. Food products comprising the compositions herein provide a natural and effective means of controlling appetite and managing weight.

### CROSS-REFERENCE TO RELATED APPLICATIONS

The present invention claims priority to U.S. Provisional Pat. App. No. 63/643,492, titled Methods and Compositions for Reducing Body Weight,” filed May 7, 2024, which is incorporated herein by reference in its entirety.

### TECHNICAL FIELD

The present invention provides methods and compositions for promoting weight loss in mammals, namely in humans and animals, such as, for example, pets. Specifically, the methods and compositions herein promote weight loss due to the production of appetite-suppressing gut hormones, increased metabolism, reduced fat and carbohydrate absorption, and reduced fat deposition through the synergistic combination of fibers and botanical extracts. Food products comprising the compositions herein provide a natural and effective means of controlling appetite and managing weight.

### BACKGROUND

The desire to maintain a healthy weight is nearly universal but many individuals face significant challenges in doing so. Indeed, obesity is one of the leading causes of premature death worldwide. In 2022, it was estimated that approximately one billion people were obese and, often, the perception of obesity carries a significant stigma. However, while many individuals desire to lose weight and maintain a healthy weight, most fail to do so or fail to maintain the weight loss long-term.

A significant factor in the failure of many to achieve and/or maintain ideal body weight typically includes managing appetite, which typically results from the regulation of certain gut hormones. Likewise, a person's rate of metabolism significantly affects the use of food energy and, consequently, the production and storage of energy as fat.

The regulation of appetite typically involves complex physiological mechanisms, including the secretion of gut hormones, such as, for example, Glucagon-like Peptide (GLP-1), Peptide YY (PYY), and Cholecystokinine (CCK). These gut hormones play a crucial role in suppressing hunger and promoting satiety.

Another factor in maintaining a healthy weight in a person relates to their rate of metabolism. It is commonly known that metabolism slows as a person ages, which may cause increased fat production and storage. Specifically, increasing metabolism helps a body preferentially utilize food calories for energy rather than for fat production and storage.

Internal metabolic mechanisms may also affect the rate of absorption of dietary fat and carbohydrates. As metabolism slows, the rate of absorption of dietary fat and carbohydrates during food intake may increase. This may also lead to undesirable and difficult-to-control weight gain.

A need, therefore, exists for improved methods and compositions for aiding a person in maintaining a healthy weight. Specifically, a need exists for improved methods and compositions that help a person regulate gut hormones to aid in managing appetite. In addition, a need exists for improved methods and compositions that increase metabolism thereby increasing the utilization of food calories for energy rather than fat storage. Moreover, a need exists for improved methods and compositions that reduce absorption of dietary fat and carbohydrates, thereby reducing calories that would otherwise be used for fat storage.

Typical weight loss treatments often include one or more of the following interventions: attempted dietary and/or lifestyle changes, medications, and/or surgeries, to name a few. Oftentimes, dietary and lifestyle changes may be expensive and often do not produce long-term effects. Likewise, medications and surgeries can also be expensive and intrusive, often having significant side effects and posing other health risks. Oftentimes, medications involve the use of synthesized molecules, where the long term effects of using such medication is poorly known.

A need, therefore, exists for improved methods and compositions that provide a natural and sustainable approach to managing appetite and promoting weight management. Specifically, a need exists for improved methods and compositions that are utilized by an individual without requiring pharmaceutical and/or surgical interventions. In addition, a need exists for improved methods and compositions that may be incorporated into a wide range of human foods, pet foods, beverages, supplement products, and other like ingestible options for consumers.

### SUMMARY OF THE INVENTION

The present invention provides methods and compositions for promoting weight loss. Specifically, the methods and compositions herein promote weight loss due to the production of appetite-suppressing gut hormones, increased metabolism, reduced fat and carbohydrate absorption, and reduced fat deposition through the synergistic combination of fibers and botanical extracts. Food products comprising the compositions herein provide a natural and effective means of controlling appetite and managing weight.

To this end, in an embodiment of the present invention, a composition for reducing body weight is provided. The composition comprises: at least one fiber selected from the group of arabinoxylan, a blend of Konjac glucomannan, xanthan gum, and alginate, and blends thereof; and at least one extract selected from the group of Caralluma fimbriata (cactus) extract, epigallocatechin gallate (EGCG), Ascophyllum nodosum extract, proteinase inhibitor II (PI2), and blends thereof.

In an embodiment, a food, beverage, or supplement comprises the composition of the present invention.

It is, therefore, an advantage and objective of the present invention to provide improved methods and compositions for aiding a person in maintaining healthy weight.

Specifically, it is an advantage and objective of the present invention to provide improved methods and compositions that help a person regulate gut hormones to aid in managing appetite.

In addition, it is an advantage and objective of the present invention to provide improved methods and compositions that increase metabolism thereby increasing the utilization of food calories for energy rather than fat storage.

Moreover, it is an advantage and objective of the present invention to provide improved methods and compositions that reduce absorption of dietary fat and carbohydrates, thereby reducing calories that would otherwise be used for fat storage.

Further, it is an advantage and objective of the present invention to provide improved methods and compositions that provide a natural and sustainable approach to managing appetite and promoting weight management.

Specifically, it is an advantage and objective of the present invention to provide improved methods and compositions that are utilized by an individual without requiring pharmaceutical and/or surgical interventions.

In addition, it is an advantage and objective of the present invention to provide improved methods and compositions that may be incorporated into a wide range of human foods, pet foods, beverages, supplement products, and other like ingestible options for consumers.

Additional features and advantages of the present invention are described in, and will be apparent from, the detailed description of the presently preferred embodiments and from the drawings.

### DETAILED DESCRIPTION OF THE PRESENTLY PREFERRED EMBODIMENTS

The present invention provides methods and compositions for promoting weight loss in mammals, preferably in human beings and pets. Specifically, the methods and compositions herein promote weight loss due to the production of appetite-suppressing gut hormones, increased metabolism, reduced fat and carbohydrate absorption, and reduced fat deposition through the synergistic combination of fibers and botanical extracts. Food products comprising the compositions herein provide a natural and effective means of controlling appetite and managing weight.

In a preferred embodiment of the present invention, compositions comprising combinations of dietary fibers and botanical extracts may be utilized to stimulate the production of appetite-suppressing gut hormones, particularly GLP-1, PYY, and CCK, in the gastrointestinal tract of both humans and animals, namely pets. The compositions may further reduce the number of dietary calories available for fat storage by the body and may further aid in increasing metabolic mechanisms within a body.

A blend of dietary fibers may preferably be utilized in compositions of the present invention. Specifically, in an embodiment, fibers such as hydrolyzed arabinoxylans, a blend of Konjac glucomannan, xanthan gum, and alginate, and Caralluma fimbriata (cactus) extract containing an amount of pregnane glycosides, may be utilized. Pregnane glycosides are naturally occurring, typically plant derived sugar conjugates of C21 steroidal compounds. The glycone part of these compounds is an oligosaccharide chain arranged in a linear fashion, rather than branched, and linked to a hydroxy group through an acetal linkage.

In a preferred embodiment, compositions of the present invention may preferably comprise one or more of Arabinoxylan, a blend of Konjac glucomannan, xanthan gum, and alginate, and/or an extract of Caralluma fimbriata (a cactus). Specifically, in an embodiment of the present invention, the compositions may comprise between about 0 g and about 60 g Arabinoxylan, more preferably between about 10 g and about 40 g Arabinoxylan, or most preferably between about 15 g and about 30 g Arabinoxylan. Moreover, compositions of the present invention may further preferably comprise between about Og and about 25 g of a fiber blend of Konjac glucomannan, xanthan gum, and alginate, more preferably, between about 2.5 g and about 20 g of the fiber blend, or most preferably, between about 7.5 g and about 15 g of the fiber blend. Preferably, the fiber blend may comprise between about 4 g and about 11 g of Konjac glucomannan, between about 1 g and 3 g of xanthan gum and between about 0.8 g and 2.5 g of alginate within the fiber blend.

In addition, compositions of the present invention may further preferably comprise between about 0 and about 15 g Caralluma fimbriata (cactus) extract, more preferably, between about 0.5 g and about 10 g Caralluma fimbriata (cactus) extract, or most preferably, between about 1 g and about 6 g Caralluma fimbriata (cactus) extract. Preferably, compositions of the present invention may comprise a blend of each of the fiber ingredients listed above in the ranges provided.

Additionally, compositions of the present invention may comprise the following botanical extracts, either alone or in a blend thereof. Specifically, in an embodiment, the compositions of the present invention may comprise between 30 mg and about 700 mg of Epigallocatechin gallate (EGCG), which may be extracted from green tea, more preferably, between about 40 mg and about 650 mg EGCG, or most preferably, between about 50 mg and about 500 mg of EGCG. In addition, compositions of the present invention may comprise between about 0 and 1000 mg of Ascophyllum nodosum (brown algae) extract comprising an amount of naturally occurring polyphenols, more preferably, between about 75 mg and about 900 mg of Ascophyllum nodosum (brown algae) extract, or most preferably, between about 100 mg and about 800 mg Ascophyllum nodosum (brown algae) extract. Moreover, compositions of the present invention may comprise between about 0 mg and 1500 mg of proteinase inhibitor II (PI2), typically extracted from potatoes, more preferably, between about 75 mg and 1350 mg PI2, or most, preferably between about 100 mg and 1200 mg PI2.

Without being bound by theory, the combination of the fibers, as described above, and the extracts, as described above, may synergistically promote the growth of beneficial gut bacteria and further may enhance the fermentation processes in the colon. As a result, there may be an increase in the production and release of appetite-suppressing gut hormones, particularly GLP-1, PYY, and/or CCK, which may help in controlling appetite and promoting feelings of fullness after eating. The combination of the botanical extracts may reduce the number of calories available for fat storage and may further increase metabolism to convert calories into energy rather than for fat storage. Thus, the present invention comprises compositions and methods of using the same for increasing lean body mass.

The compositions described herein may be incorporated into various foods and beverages that may be utilized as effective delivery systems for the compositions to the gut in mammals, namely humans and/or animals, such as, for example, pets. Examples of foods and beverages may include, but may are not limited to, cereals and granola bars, bread and other baked goods, dairy products, such as yogurt, for example, beverages such as smoothies, shakes, and other like beverages, snack items, such as, for example, protein bars, crackers, cookies, and other like snack items, pet foods and snacks, including kibble and wet or dry pet food pellets, and supplements, which may be in the form of capsules, tablets, powders, liquids, gels, or other like dietary supplements.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

We claim:

1. A composition for reducing body weight in mammals comprising:
at least one fiber selected from the group consisting of arabinoxylan, a blend of Konjac glucomannan, xanthan gum, and alginate, and blends thereof; and
at least one extract selected from the group consisting of Caralluma fimbriata (cactus) extract, epigallocatechin gallate (EGCG), Ascophyllum nodosum extract, proteinase inhibitor II (PI2), and blends thereof.

2. A food, beverage, or supplement comprising the composition of claim 1.
